(19) United States
(12) Patent Application Publication
(10) Pub. No.: US 2016/0074174 A1
(43) Pub. Date: Mar. 17, 2016

(54) IBD Expandable Ti

(71) Applicant: Nexus Spine, LLC, Salt Lake City, UT (US)

(72) Inventors: Peter Halverson, Draper, UT (US); David Hawkes, Pleasant Grove, UT (US)

(21) Appl. No.: 14/854,019

(22) Filed: Sep. 14, 2015

Related U.S. Application Data
(60) Provisional application No. 62/050,038, filed on Sep. 12, 2014.

(51) Int. Cl. A61F 2/44

(52) U.S. Cl. CPC A61F 2/4455; A61F 2002/4415; A61F 2002/448; A61F 2002/4687

(57) ABSTRACT

An expandable interbody spacer for use in spinal fusion procedures includes a plurality of rigid segments connected by flexible connections to form a ring encompassing and defining a hollow central area of variable dimensions. The flexible connections between the plurality of rigid segments may include flexible regions formed between the rigid segments or a continuous flexible member extending along a multisegmented region. The flexible regions formed between the rigid segments may be integrally formed with the rigid segments. One or more of the flexible regions formed between the rigid segments may include a plurality of flexure divisions extending between adjacent rigid segments. One or more of the flexible regions formed between the rigid segments may include a flexure extending between adjacent rigid segments. The rigid segments may include surfaces to limit the range of motion between adjoining rigid segments.

### CROSS-REFERENCE TO RELATED APPLICATIONS

This application claims the benefit of U.S. Provisional Application No. 62/050,038, filed Sep. 12, 2014.

### BACKGROUND OF THE INVENTION

1. Field of the Invention

The present invention relates to spinal fusion, and more particularly to an expanding interbody spacer for use in spinal fusion procedures.

2. Background and Related Art

Spinal fusion is a surgical procedure used to correct problems with the vertebrae in the spine. Spinal fusion is used to fuse or rigidly join two or more adjacent vertebrae so that they heal into a single solid bone. One general type of spinal fusion involves removing the intervertebral disc. When the disc space has been cleared out, a metal, plastic, or bone spacer is implanted between the adjoining vertebrae in the space previously occupied by the intervertebral disc. The spacers or cages often contain bone graft material to promote bone healing and to facilitate fusion. Once the spacer or cage is in place, surgeons often use screws, plates, and/or rods to further stabilize the spine.

Interbody fusion may be performed using a variety of different approaches. These approaches may be visualized with respect to FIG. 1, which shows a horizontal cross-sectional view of a human trunk, showing an intervertebral disc and surrounding structures. Recognized approaches include anterior lumbar interbody fusion (ALIF), lateral lumbar interbody fusion, including extreme lateral interbody fusion (XLIF) and direct lateral interbody fusion (DLIF), posterior lumbar interbody fusion (PLIF), and transforaminal lumbar interbody fusion (TLIF). FIG. 1 shows a general direction of approach for several of these options. Each option provides certain recognized benefits and challenges.

For example, the ALIF approach (illustrated by FIGS. 2-5) provides a benefit of allowing for implantation of an interbody spacer having a large footprint, such as from 30-38 mm by 26-30 mm. Such an implant can be stable as a standalone device (i.e., the need for additional support such as posterior screws and rods is lessened), and serves to prevent or reduce post-operative subsidence of the interbody spacer. The ALIF approach also provides a large graft window, which can accelerate boney union and ease access and placement of the interbody spacer and graft materials. However, the ALIF procedure involves significant mobilization of soft tissues, as the spine is approached through the abdomen as illustrated in FIGS. 2-3. This involves retraction of abdominal muscles and the peritoneum and displacement of large blood vessels (abdominal aorta, inferior vena cava, iliac artery, and/or iliac vein). Additionally, the anterior longitudinal ligament must be cut as part of the surgery, which may further destabilize the spine. The necessary techniques may result in higher levels of blood loss as well as other unwanted side effects such as retrograde ejaculation. Furthermore, a second surgeon (e.g., a vascular surgeon) is often required, with accompanying increased costs. Because the implant is larger, there are increased costs of manufacture for the device. Finally, when additional stability is desired, the patient must be repositioned for posterior access for placement of posterior screws, etc. via additional surgery.

The lateral approaches (illustrated by FIGS. 6-9) provide benefits of allowing implantation of an interbody spacer having a large footprint, such as from 45-55 mm by 18-26 mm. Such implants are stable as standalone devices and prevent or reduce subsidence as discussed above. The lateral approaches also provide a large graft window, which can accelerate boney union, and can be performed by a single surgeon. The lateral approaches, however, are limited by issues of access, with access to the L4-L5 interbody space being difficult and access to the L5-S1 space being impossible. One of the difficulties with such surgery is the potential for damage to nerves disturbed during surgery, with significant numbers of patients reporting leg pain six to twelve months after surgery. Additionally, subsidence issues remain, and where additional stability is to be achieved through placement of posterior screws, etc., the patient also must be repositioned for posterior access.

The PLIF approach (illustrated by FIGS. 10-13) provides a benefit of avoiding repositioning of the patient for placement of pedicle screws/rods/plates and provides ease of access to the spine for surgical placement of the interbody spacer. However, using conventional techniques and implants, the posterior approach involves challenges of mobilization of the spinal cord, a small graft window, and conventionally only small interbody spacers such as from 10-11 mm can be placed through the small graft window. The small interbody spacer size generally means that the interbody spacer cannot serve as a standalone implant: additional support through posterior pedicle screws, rods, and/or plates is generally required. Furthermore, the placement and implant size restrictions inherent in the posterior approach generally results in the interbody spacer being placed at locations of less dense bone. Other involved risks include failure to achieve proper lordosis (e.g., due to being placed at a location of lower bone density) and risks associated with any necessary laminectomy associated with the procedure.

The TLIF approach (illustrated by FIGS. 14-15) provides benefits of avoiding repositioning of the patient for placement of pedicle screws/rods/plates for posterior stabilization and provides ease of access with only minimal nerve root retraction necessary. However, using conventional techniques and implants, the TLIF approach involves challenges of mobilization of the spinal cord, a small graft window, and conventionally only small interbody spacers such as from 10-11 mm can be placed through the small graft window. The small interbody spacer size generally means that the interbody spacer cannot serve as a standalone implant: additional support through posterior pedicle screws, rods, and/or plates is generally required. Furthermore, placement and implant size restrictions inherent in the posterior approach generally results in the interbody spacer being placed at locations of less dense bone. Other involved risks include failure to achieve proper lordosis (e.g., due to being placed at a location of lower bone density) and risks associated with any necessary laminectomy associated with the procedure.

Final stability in spinal fusion surgery is most often achieved by spanning the intervertebral disc space with an implanted interbody spacer. Furthermore, fusion may occur more rapidly when the implant is loaded with osteoconductive and/or osteoinductive materials, and larger implants allow for larger volumes of such materials. The desire for larger implants must be balanced, however, with considerations of surgical access. Generally, it is desirable to minimize the surgical window to minimize the trauma to the patient and soft tissue, to ease insertion for the surgeon, and to speed recovery. Conventionally, each of the approaches discussed above involves tradeoffs between implant size and the surgical access window. No current device has allowed for a maximal implant size to be implanted with minimal surgical access.

Certain predicate devices have attempted to increase the height and/or lordosis of the device after implantation. The complexity of those devices often results in increased manufacturing costs, increased likelihood of failure, and complicated surgical techniques. Other predicate devices have attempted to increase the footprint of the device. The complexity of those devices has resulted in increased manufacturing costs, increased likelihood of failure, and complicated surgical techniques without a significant increase in implanted footprint.

For these and other reasons, there remain unaddressed needs in the area of implanted interbody spacers for use in spinal fusion procedures.

### BRIEF SUMMARY OF THE INVENTION

Implementation of the invention provides an expandable interbody spacer capable of being used in minimally invasive spinal fusion procedures such as PLIF and TLIF while providing a final implant size more commonly in use with more-invasive spinal fusion procedures such as ALIF, XLIF, and DLIF. Additionally implementation of the invention can also be used to minimize the trauma of ALIF, XLIF, or DLIF surgical approaches by minimizing the needed surgical window. Implementation of the invention also provides methods for manufacturing such interbody spacers and methods for using such interbody spacers.

According to implementations of the invention, an expandable interbody spacer for use in spinal fusion procedures includes a plurality of rigid segments connected by flexible connections to form a ring encompassing and defining a hollow central area of variable dimensions. The flexible connections between the plurality of rigid segments may include flexible regions formed between the rigid segments. The flexible regions formed between the rigid segments may be integrally formed with the rigid segments. One or more of the flexible regions formed between the rigid segments may include a plurality of flexure divisions extending between adjacent rigid segments. One or more of the flexible regions formed between the rigid segments may include a flexure extending between adjacent rigid segments.

In some implementations, a rigid segment adjacent the flexure may include one or more surfaces adapted to define a maximum range of motion between the adjacent rigid segments. The maximum range of motion between the adjacent rigid segments may include between approximately twenty and approximately one hundred and twenty degrees.

The flexible connections between the plurality of rigid segments may include a continuous flexible member extending between and forming multiple flexible connections of the expandable interbody spacer. The continuous flexible member may pass through one or more channels formed in one or more of the rigid segments. The continuous flexible member may extend around the entire interbody spacer.

The rigid segments may include stability channels adapted to receive a stability rod or a plurality of stability rods therein, whereby insertion of the stability rod therein serves to limit motion between adjacent rigid segments. One or more of the rigid segments may include a conical contact surface adapted to contact an adjacent rigid segment. One or more of the rigid segments may include a cylindrical surface adapted to contact an adjacent rigid segment. The ring may be adapted such that the hollow central area can be made narrow during initial insertion of the interbody spacer and then expanded horizontally as the interbody spacer is fully inserted and placed in an intervertebral space between adjoining vertebrae. The rigid segments may include a pull channel adapted to permit application of a pulling force to the interbody spacer to expand the interbody spacer.

As described, the interbody spacer encompasses and defines a hollow central area. The hollow central area is adapted to receive a material therein after implantation. The material placed after implantation may include a bone graft material. Alternatively or additionally, the material may include an osteoinductive material. Alternatively or additionally, the material may include an osteoconductive material. These materials may assist in the formation of bone after implantation to fuse together the vertebrae adjacent the interbody spacer.

According to further implementations of the invention, an expandable interbody spacer for use in spinal fusion procedures includes a plurality of rigid segments arranged to form a ring defining a hollow central area and a plurality of flexible connections formed by flexible members extending between adjacent rigid segments such that the ring can be deformed to modify dimensions of the hollow central area. The flexible members formed between the rigid segments may be integrally formed with the rigid segments. One or more of the flexible regions formed between the rigid segments may include a plurality of flexure divisions extending between adjacent rigid segments.

According to additional implementations of the invention, an expandable interbody spacer for use in spinal fusion procedures includes a plurality of rigid segments arranged to form a ring defining a hollow central area and a flexible member extending around the ring and between adjacent rigid segments such that the ring can be deformed to modify dimensions of the hollow central area.

According to additional implementations of the invention, expandable interbody spacers as described above are manufactured with flexible regions formed between rigid segments to form a ring. The expandable interbody spacer may be manufactured in a partially deployed position to reduce resultant stresses on the interbody spacer's flexible regions during insertion and during deployment. According to additional implementations of the invention, expandable interbody spacers as described above are manufactured as individual rigid segments that are then connected by one or more flexible members to form a ring. For example, the rigid segments may be inserted onto a flexible member such as a nitinol wire.

According to additional implementations of the invention, methods are provided for placing expandable interbody spacers as described above are in the intervertebral space between vertebrae. According to such methods, an interbody spacer is compressed such that the space encompassed by the interbody spacer is long and thin. Thereafter or essentially simultaneously, one or more insertion rods are inserted into one or more corresponding stability channels to maintain the interbody spacer in this compressed state and to essentially eliminate rotation of adjacent segments relative to each other at the joints where the insertion rod or rods is/are present.

The interbody spacer is introduced at the surgical site with the insertion rod or rods present in the stability channel or channels, and is inserted through a surgical incision into the previously cleared interbody space until a distal portion of the interbody spacer is within the interbody space. At that point, the insertion rod or insertion rods is/are partially removed, which allows the distal portion to expand laterally, either naturally due to tensions in the joints of the implant or under forces applied by a pull rod. The lateral expansion of the distal portion of the implant allows additional room for further insertion of the implant, and the process proceeds with additional insertion, further removal of the insertion rod, and further expansion of the implant until the implant is completely located within the interbody space and is fully expanded therein. Once the implant is properly placed, the hollow area defined and encompassed by the implant may be filled with any desired material as described more fully herein.

### BRIEF DESCRIPTION OF THE SEVERAL VIEWS OF THE DRAWINGS

The objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 shows a sectional view of a lumbar region of the human spine showing directions for various spinal interbody fusion approaches;

FIGS. 2-5 show views of access and implants typically used for an anterior lumbar interbody fusion (ALIF) approach;

FIGS. 6-9 show views of access and implants typically used for lateral lumbar interbody fusion (XLIF and DLIF) approaches;

FIGS. 10-13 show views of access and implants typically used for posterior lumbar interbody fusion (PLIF) approaches;

FIGS. 14-15 show views of access and implants typically used for transforaminal lumbar interbody fusion (TLIF) approaches);

FIG. 16 shows a plan view of an embodiment of a multisegmented interbody spacer;

FIG. 17 shows a perspective view of an embodiment of a multisegmented interbody spacer;

FIG. 18 shows a perspective view of segments of an embodiment of a multisegmented interbody spacer;

FIG. 19 shows a perspective view of a joint of an embodiment of a multisegmented interbody spacer;

FIG. 20 shows a plan view of an alternative joint of an embodiment of a multisegmented interbody spacer;

FIG. 21 shows a perspective view of segments of an embodiment of a multisegmented interbody spacer;

FIG. 22 shows a perspective view of an embodiment of a multisegmented interbody spacer in a compressed insertion aspect;

FIG. 23 shows a plan or cross-sectional view of an embodiment of a multisegmented interbody spacer;

FIG. 24 shows plan or cross-sectional views of an embodiment of a multisegmented interbody spacer with insertion rods inserted therein;

FIG. 25 shows a perspective view of selected rigid segments of a multisegmented interbody spacer showing pull channels therein;

FIG. 26 shows an overlaid plan view of an embodiment of a multisegmented interbody spacer in partially and fully deployed states;

FIG. 27 shows an overlaid plan view of an embodiment of a multisegmented interbody spacer in partially and fully deployed states;

FIGS. 28 and 29 show perspective views of an embodiment of a multisegmented interbody spacer that has been partially disassembled;

FIG. 30 shows a perspective view of an embodiment of a multisegmented interbody spacer; and

FIG. 31 shows a side (lateral) view of an embodiment of a multisegmented interbody spacer.

### DETAILED DESCRIPTION OF THE INVENTION

A description of embodiments of the present invention will now be given with reference to the Figures. It is expected that the present invention may take many other forms and shapes, hence the following disclosure is intended to be illustrative and not limiting, and the scope of the invention should be determined by reference to the appended claims.

Embodiments of the invention provide an expandable interbody spacer capable of being used in minimally invasive spinal fusion procedures such as PLIF and TLIF while providing a final implant size more commonly in use with more-invasive spinal fusion procedures such as ALIF, XLIF, and DLIF. Additionally embodiments of the implant can also be used to minimize the trauma of ALIF, XLIF, or DLIF surgical approaches by minimizing the needed surgical window. Embodiments of the invention also provide methods for manufacturing such interbody spacers and methods for using such interbody spacers.

According to embodiments of the invention, an expandable interbody spacer for use in spinal fusion procedures includes a plurality of rigid segments connected by flexible connections to form a ring encompassing and defining a hollow central area of variable dimensions. The flexible connections between the plurality of rigid segments may include flexible regions formed between the rigid segments. The flexible regions formed between the rigid segments may be integrally formed with the rigid segments. One or more of the flexible regions formed between the rigid segments may include a plurality of flexure divisions extending between adjacent rigid segments. One or more of the flexible regions formed between the rigid segments may include a flexure extending between adjacent rigid segments.

In some embodiments, a rigid segment adjacent the flexure may include one or more surfaces adapted to define a maximum range of motion between the adjacent rigid segments. The maximum range of motion between the adjacent rigid segments may include between approximately thirty and approximately one hundred and twenty degrees.

The flexible connections between the plurality of rigid segments may include a continuous flexible member extending between and forming multiple flexible connections of the expandable interbody spacer. The continuous flexible member may pass through one or more channels formed in one or more of the rigid segments. The continuous flexible member may extend around the entire interbody spacer.

The rigid segments may include stability channels adapted to receive a stability rod therein, whereby insertion of the stability rod therein serves to limit motion between adjacent rigid segments. One or more of the rigid segments may include a conical contact surface adapted to contact an adjacent rigid segment. One or more of the rigid segments may include a cylindrical surface adapted to contact an adjacent rigid segment. The ring may be adapted such that the hollow central area can be made narrow during initial insertion of the interbody spacer and then expanded horizontally as the interbody spacer is fully inserted and placed in an intervertebral space between adjoining vertebrae. The rigid segments may include a pull channel adapted to permit application of a pulling force to the interbody spacer to expand the interbody spacer.

As described, the interbody spacer encompasses and defines a hollow central area. The hollow central area is adapted to receive a material therein after implantation. The material placed after implantation may include a bone graft material. Alternatively or additionally, the material may include an osteoinductive material. Alternatively or additionally, the material may include an osteoconductive material. These materials may assist in the formation of bone after implantation to fuse together the vertebrae adjacent the interbody spacer.

According to further embodiments of the invention, an expandable interbody spacer for use in spinal fusion procedures includes a plurality of rigid segments arranged to form a ring defining a hollow central area and a plurality of flexible connections formed by flexible members extending between adjacent rigid segments such that the ring can be deformed to modify dimensions of the hollow central area. The flexible members formed between the rigid segments may be integrally formed with the rigid segments. One or more of the flexible regions formed between the rigid segments may include a plurality of flexure divisions extending between adjacent rigid segments.

According to additional embodiments of the invention, an expandable interbody spacer for use in spinal fusion procedures includes a plurality of rigid segments arranged to form a ring defining a hollow central area and a flexible member extending around the ring and between adjacent rigid segments such that the ring can be deformed to modify dimensions of the hollow central area.

According to additional embodiments of the invention, expandable interbody spacers as described above are manufactured with flexible regions formed between rigid segments to form a ring. The expandable interbody spacer may be manufactured in a partially deployed position to reduce resultant stresses on the interbody spacer's flexible regions during insertion and during deployment. According to additional embodiments of the invention, expandable interbody spacers as described above are manufactured as individual rigid segments that are then connected by one or more flexible members to form a ring. For example, the rigid segments may be inserted onto a flexible member such as a nitinol wire.

According to additional embodiments of the invention, methods are provided for placing expandable interbody spacers as described above are in the intervertebral space between vertebrae. According to such methods, an interbody spacer is compressed such that the space encompassed by the interbody spacer is long and thin. Thereafter or essentially simultaneously, one or more insertion rods are inserted into one or more corresponding stability channels to maintain the interbody spacer in this compressed state and to essentially eliminate rotation of adjacent segments relative to each other at the joints where the insertion rod or rods is/are present.

The interbody spacer is introduced at the surgical site with the insertion rod or rods present in the stability channel or channels, and is inserted through a surgical incision into the previously cleared interbody space until a distal portion of the interbody spacer is within the interbody space. At that point, the insertion rod or insertion rods is/are partially removed, which allows the distal portion to expand laterally, either naturally due to tensions in the joints of the implant or under forces applied by a pull rod. The lateral expansion of the distal portion of the implant allows additional room for further insertion of the implant, and the process proceeds with additional insertion, further removal of the insertion rod, and further expansion of the implant until the implant is completely located within the interbody space and is fully expanded therein. Once the implant is properly placed, the hollow area defined and encompassed by the implant may be filled with any desired material as described more fully herein.

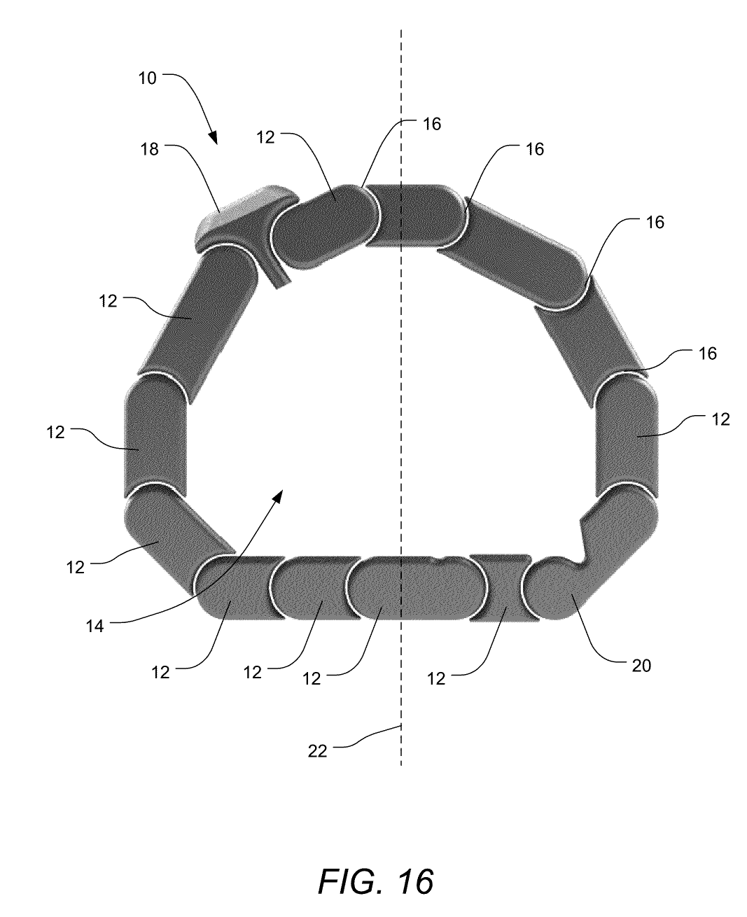

FIG. 16 shows a plan view of an exemplary embodiment of an expandable interbody spacer 10. The interbody spacer 10 includes a plurality of rigid segments 12. The rigid segments 12 form a ring encompassing and defining a hollow area 14 that is adapted to receive a material therein. For example, after the interbody spacer 10 is implanted, the surgeon may place one or more materials therein, including bone graft materials such as morcelized bone and/or bone marrow, osteoinductive materials, and/or osteoconductive materials, as is known in the art of spinal fusion implants to encourage and facilitate bone growth into and around the implant to hasten and encourage fusion of the adjacent vertebrae.

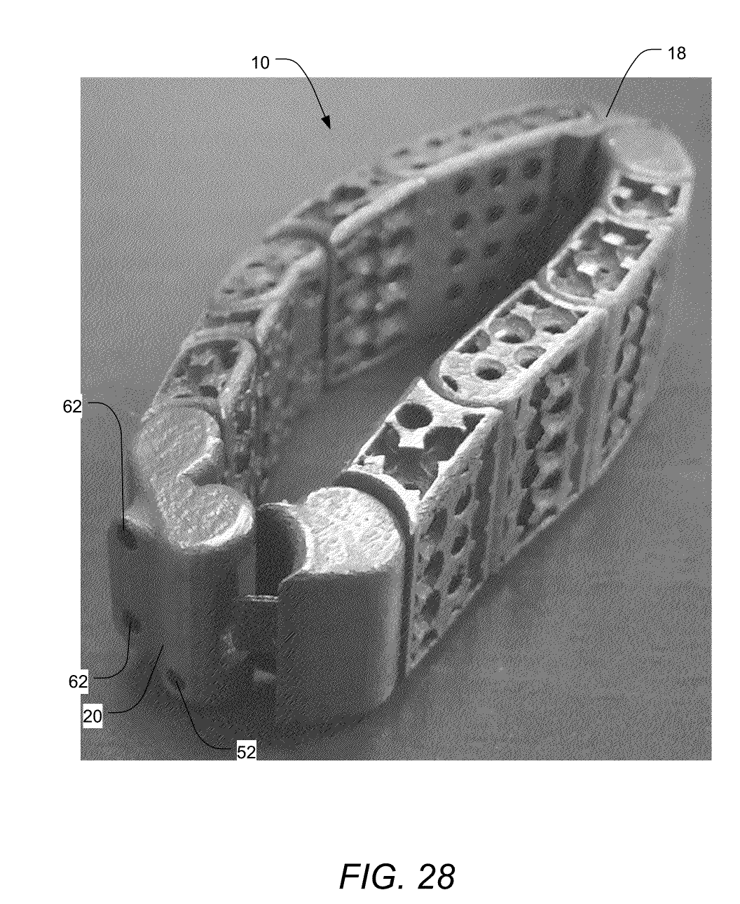

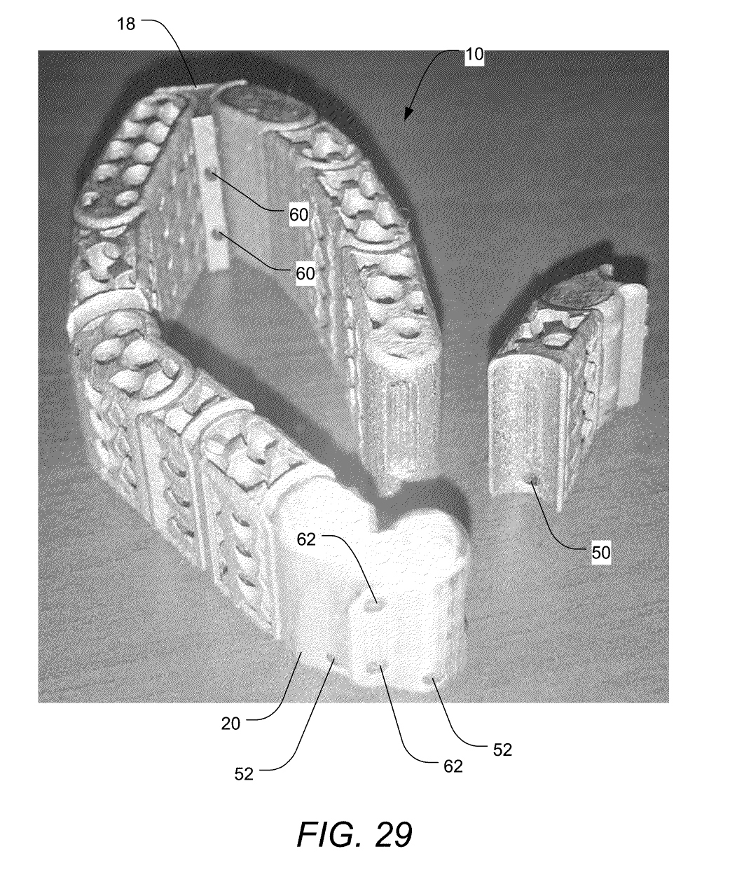

The rigid segments 12 may be formed out of materials commonly used for spinal fusion implants, including metals such as titanium, bio-compatible polymers, allograft materials, and/or a variety of natural and/or synthetic materials as is currently known in the art, and the rigid segments 12 may be manufactured or formed using conventional techniques known in the art for manufacturing implants using the selected material or materials. To the extent that conventional known implant materials are used to form the rigid segments 12, the term “rigid” is to be understood to refer to a level of rigidity achieved by manufacturing rigid segments 12 of dimensions and shapes illustrated and discussed herein using such materials. Additionally, the rigid segments 12 may also be formed of any material that may come to be used in spinal implants in the future, and the term “rigid” shall encompass a level of rigidity achieved by manufacturing rigid segments 12 of dimensions and shapes illustrated and discussed herein using such materials. The rigid segments 12 may be formed to be largely solid or may be manufactured to have varying amounts of empty space to achieve desired manufacturing and performance characteristics and/or to permit integration of bone into hollow areas defined by the rigid segments 12. Thus, some or all of the rigid segments 12 may have, for example, a honeycomb appearance, as illustrated by FIGS. 28-29. The term “rigid” therefore encompasses varying levels of rigidity depending on the amount of solidity of the rigid segments 12. Furthermore, the term “rigid” should be understood as comparative to the rigidity of linkages or joints between adjacent rigid segments 12 as illustrated and discussed herein.

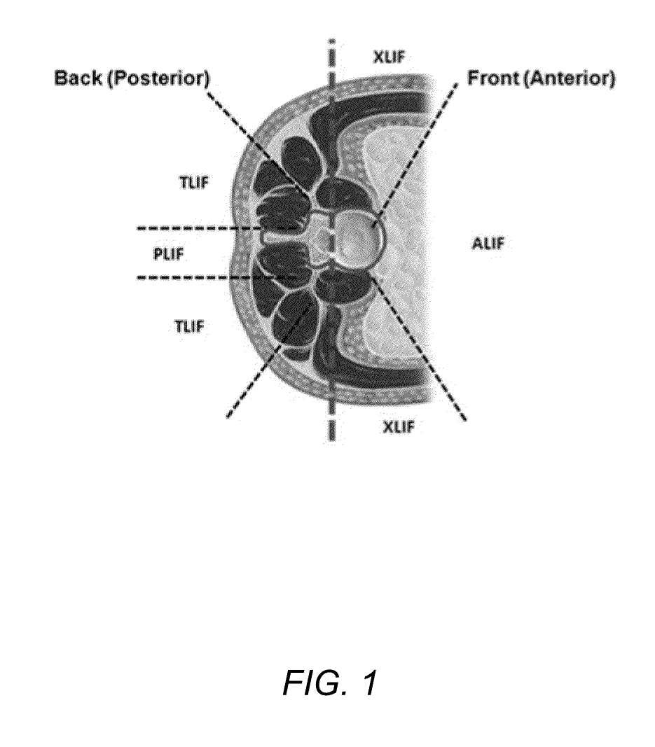

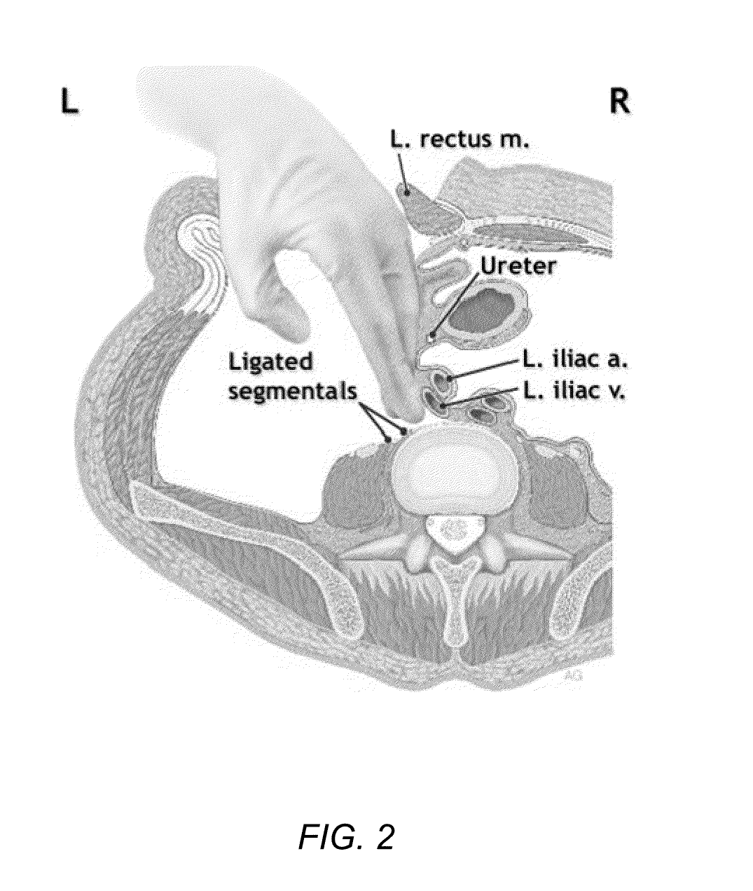

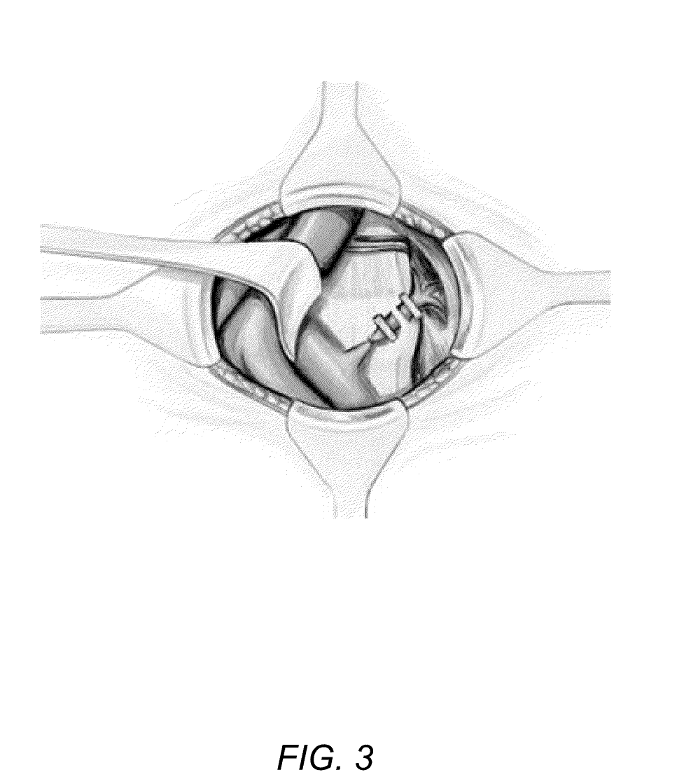

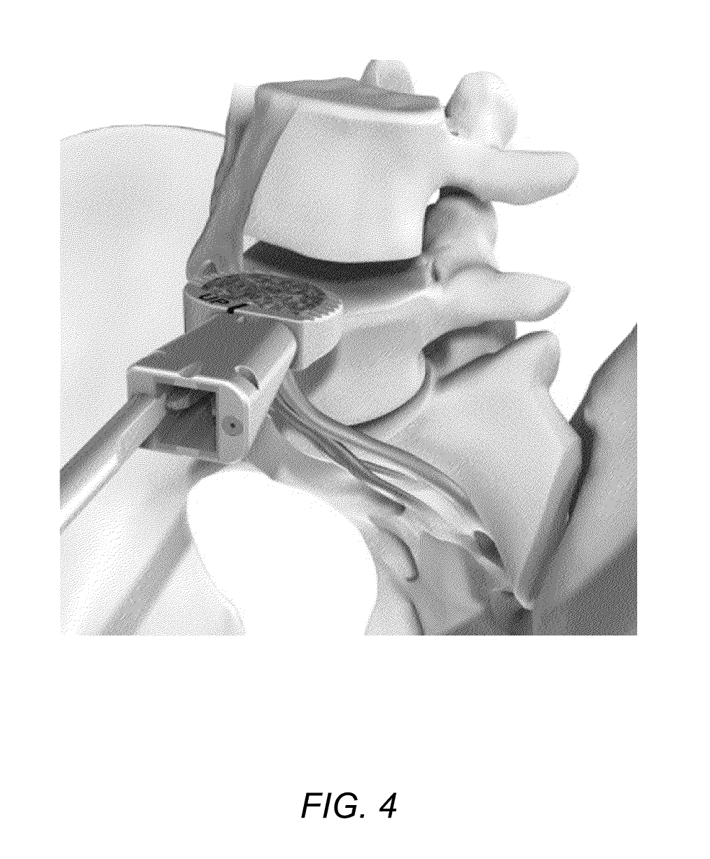

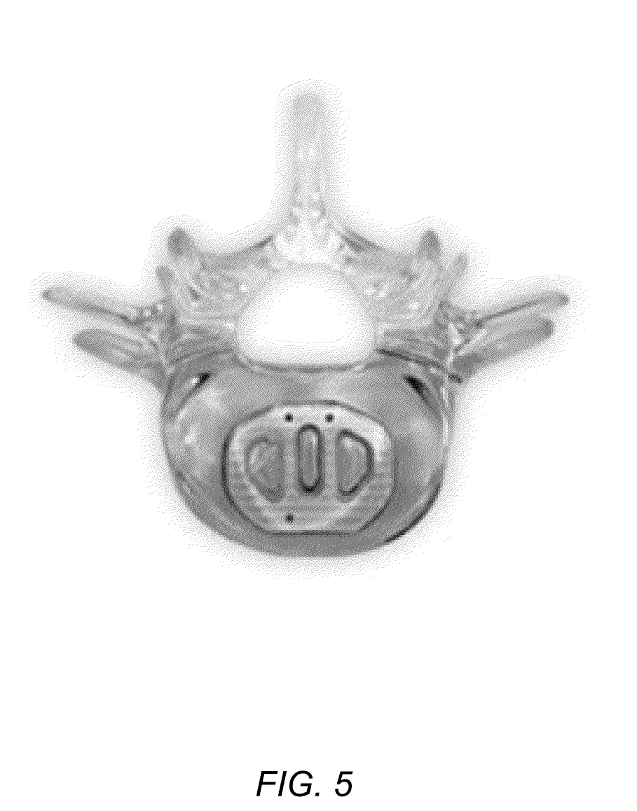

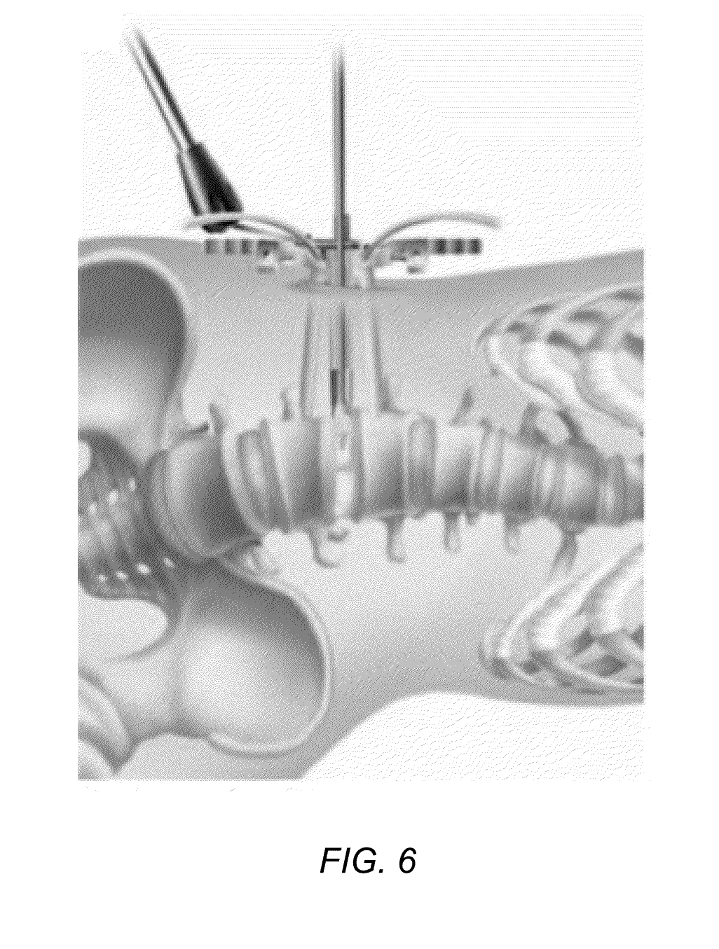

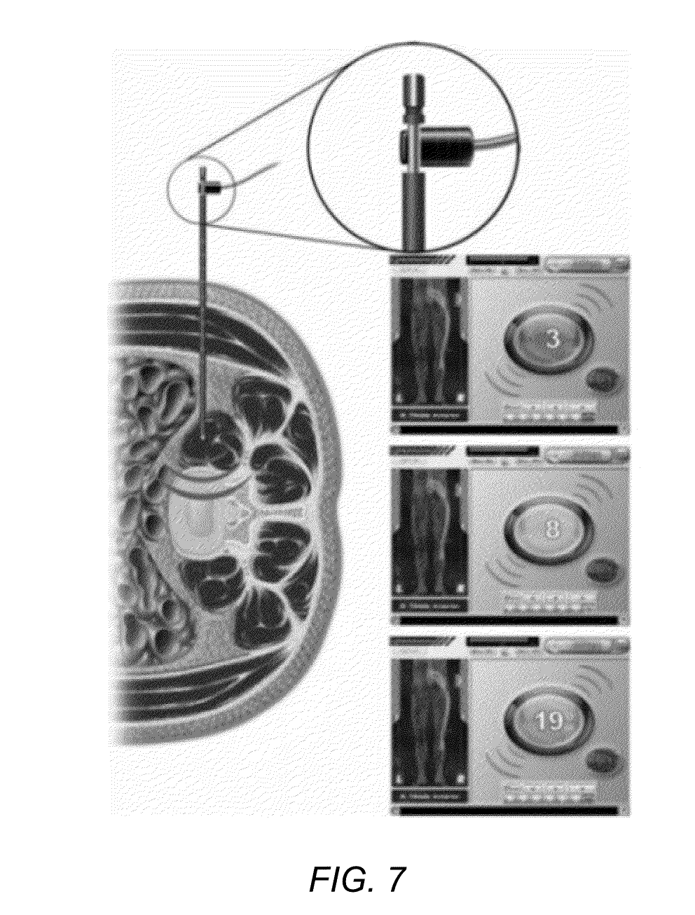

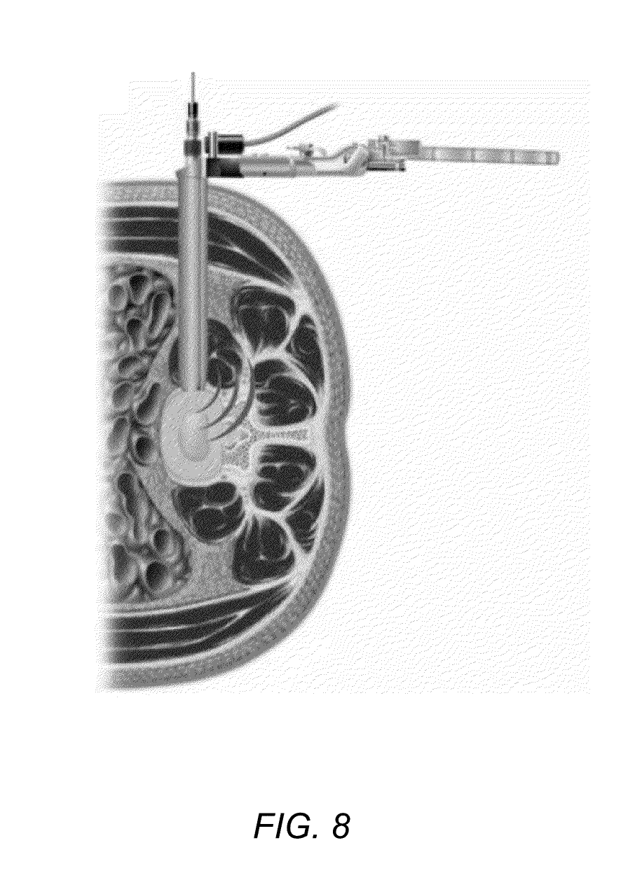

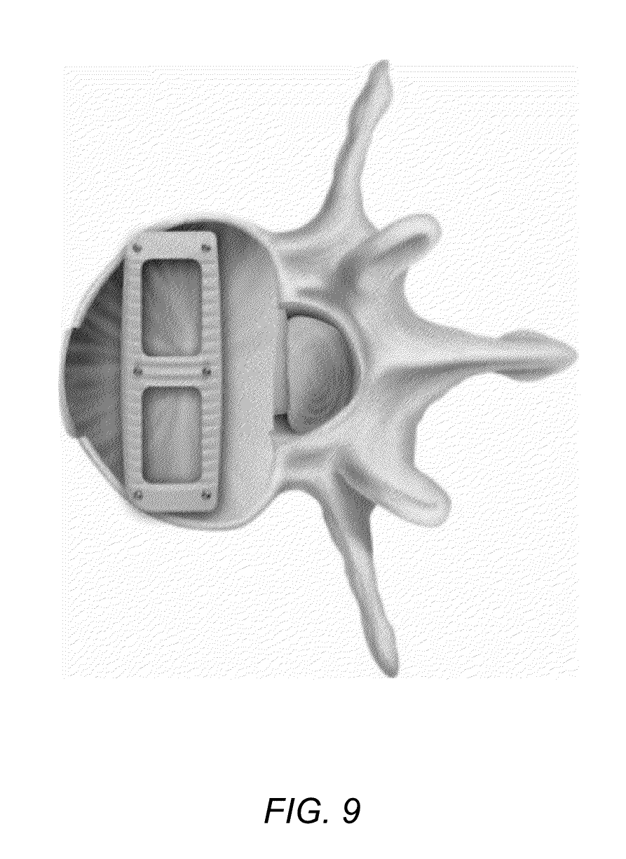

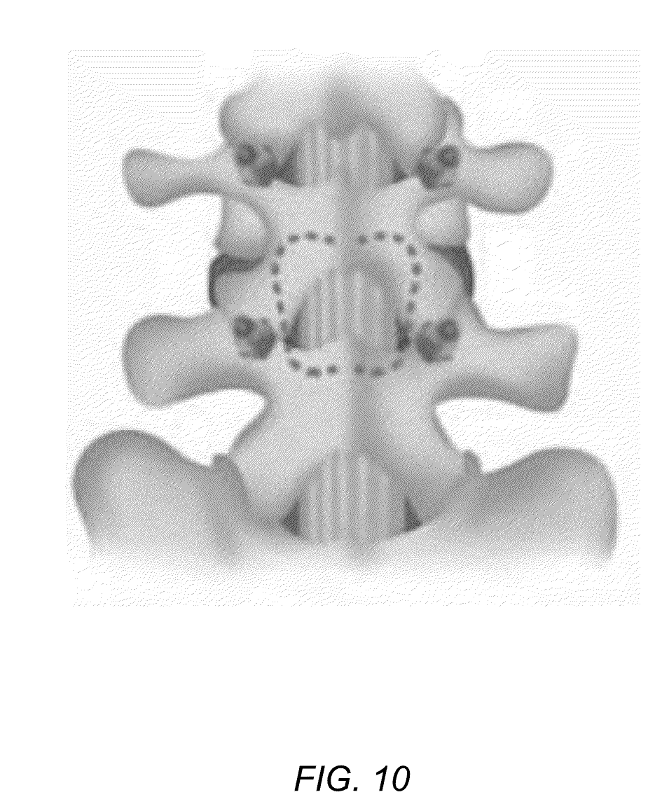

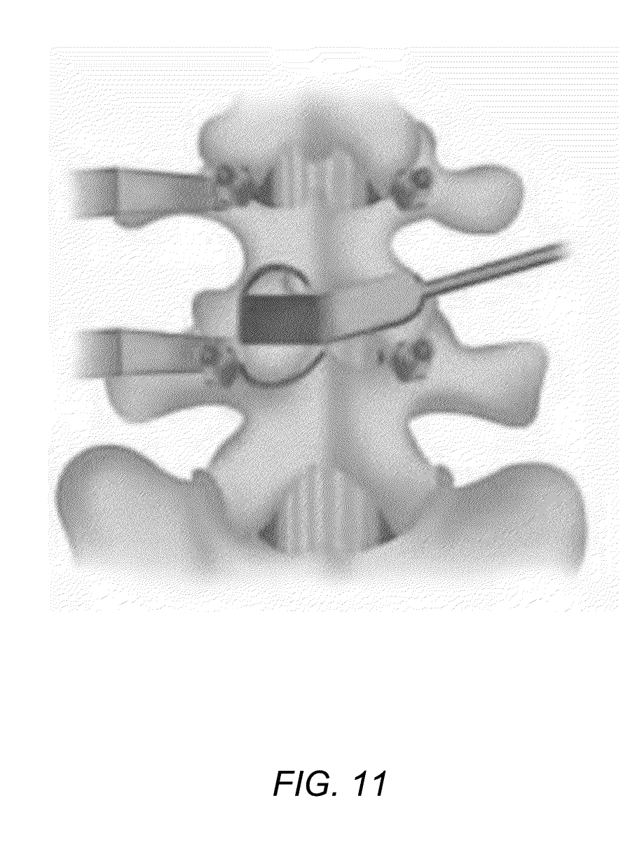

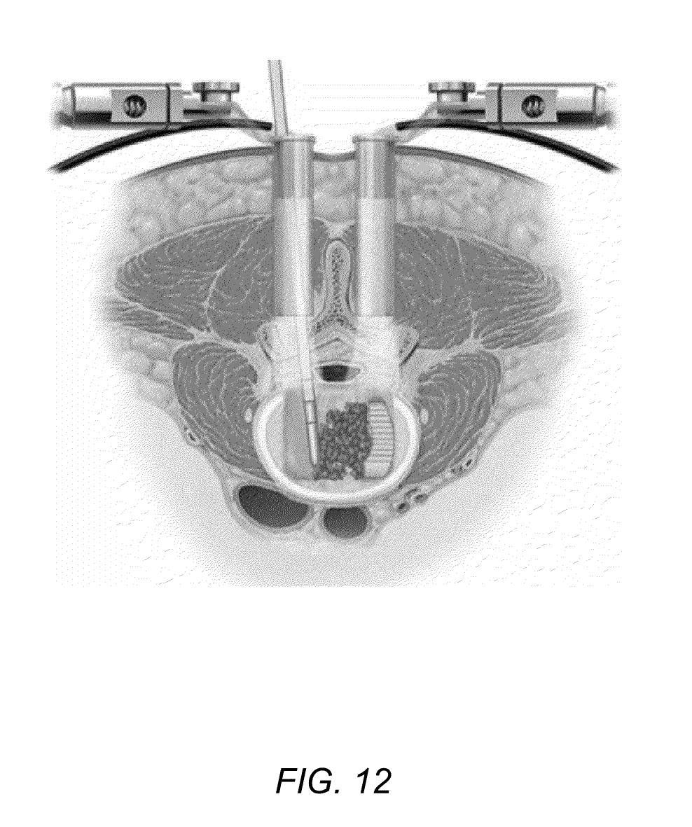

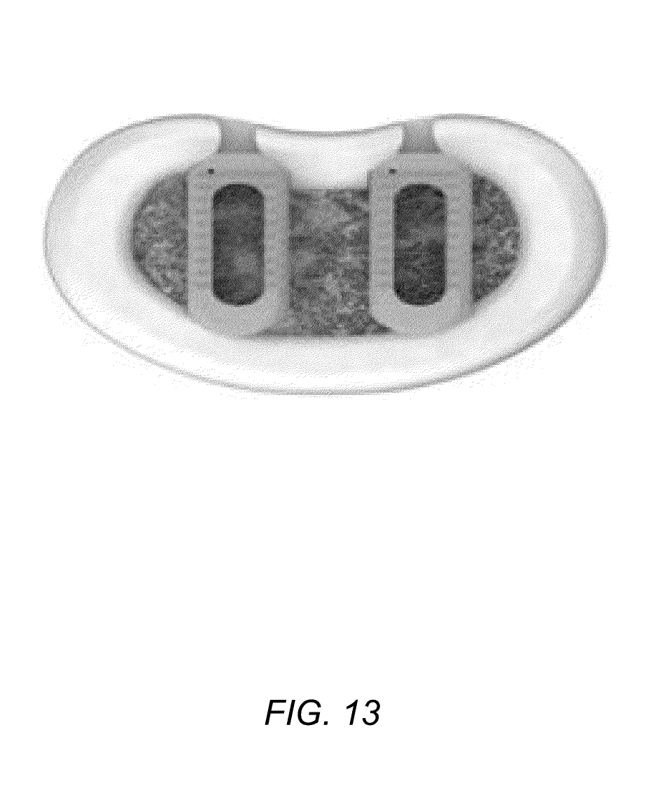

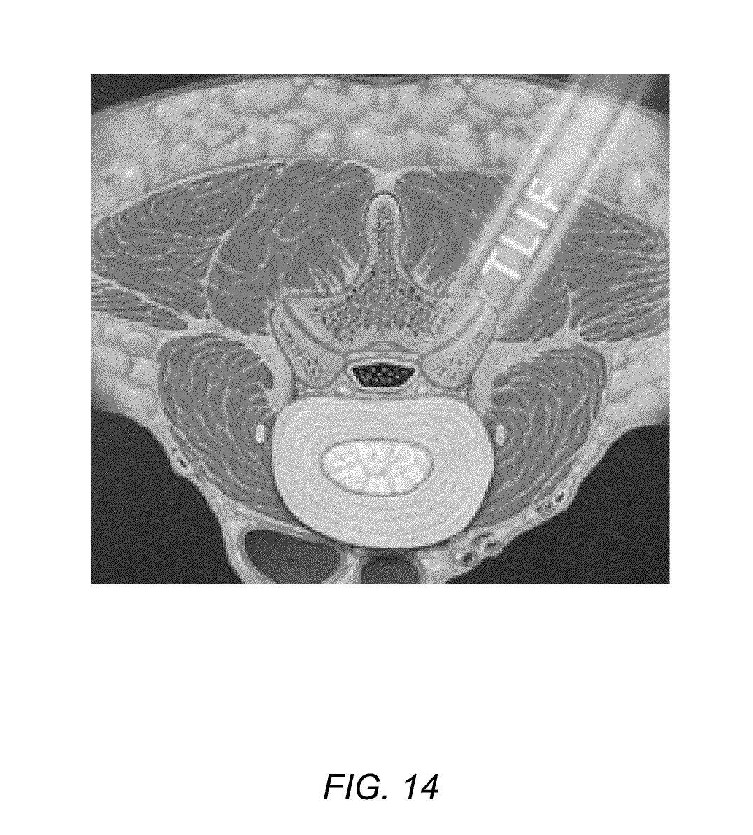

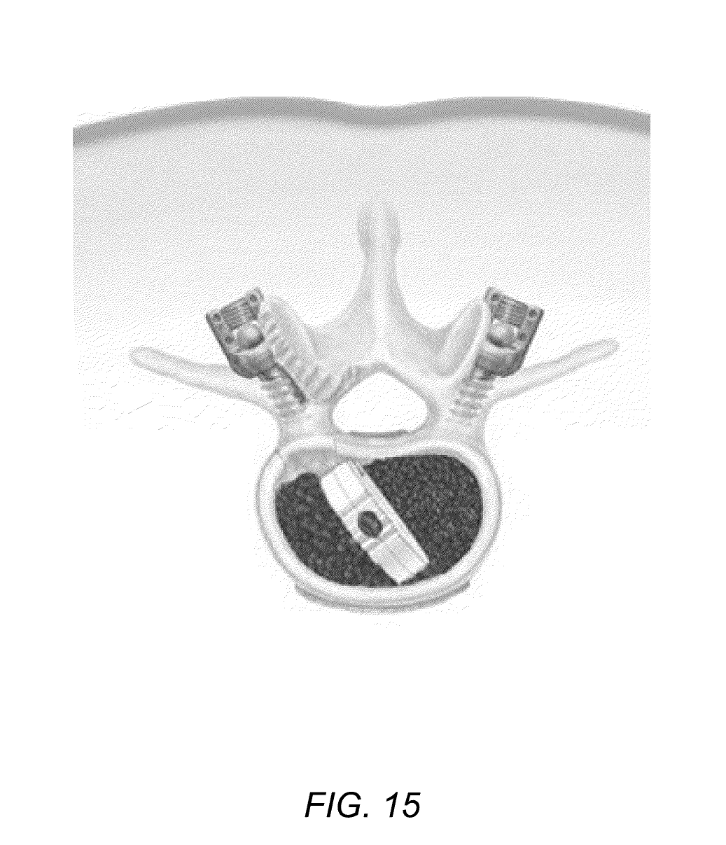

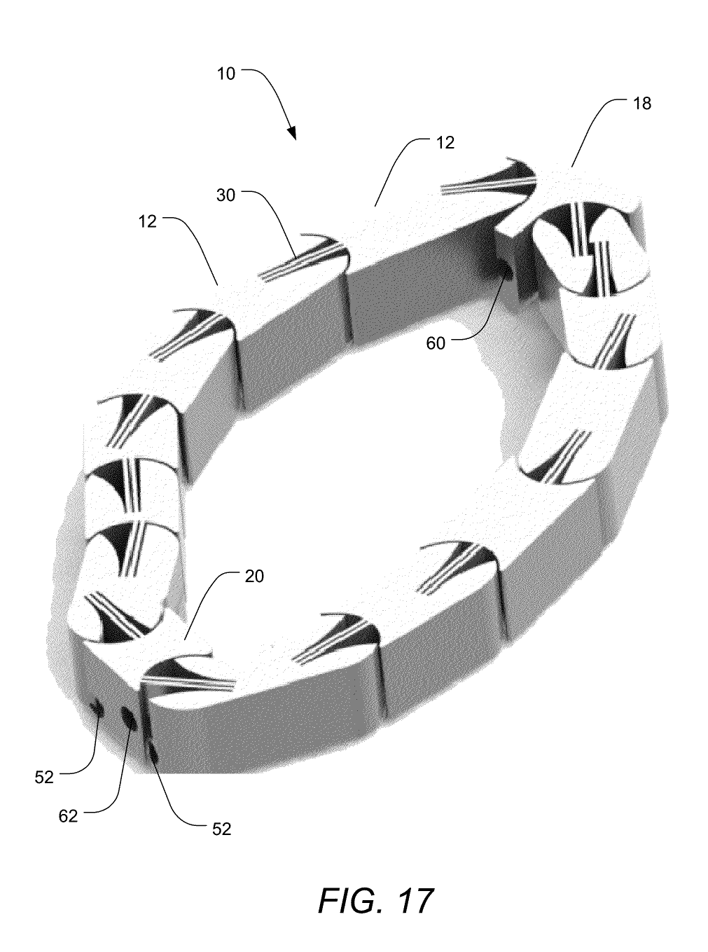

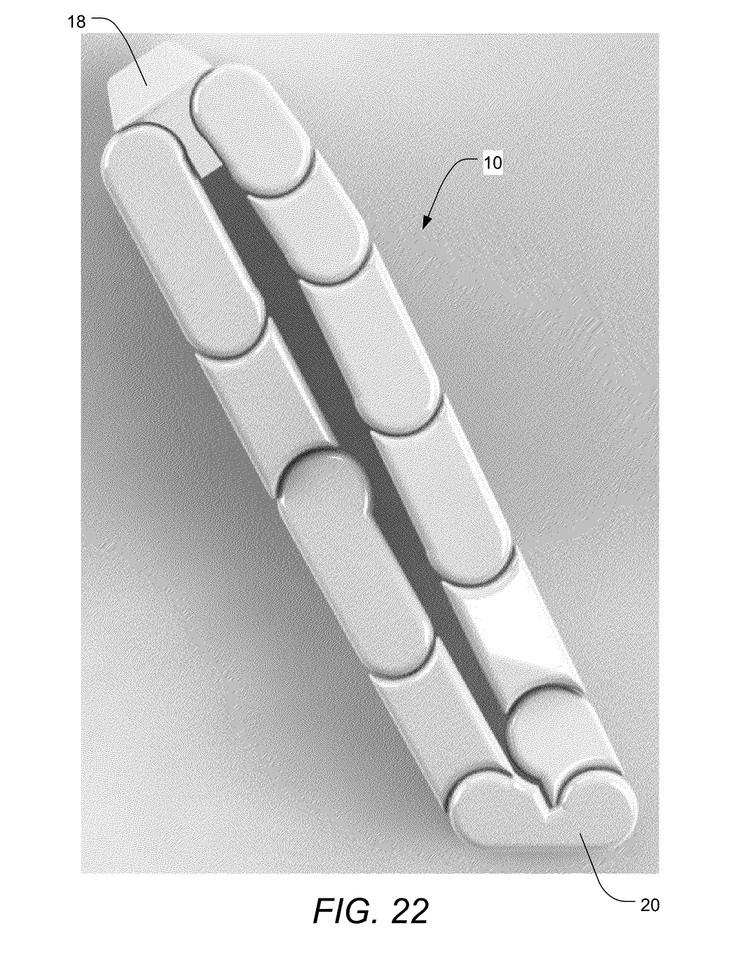

Adjacent rigid segments 12 meet at joints 16. (Not all rigid segments 12 or joints 16 are labeled in FIG. 1.) The joints 16 are relatively flexible as compared to the rigid segments 12. The joints 16 permit the interbody spacer 10 to be flexed and compressed from an expanded position shown in FIG. 16 to a narrow compressed position as shown in, for example, FIG. 22, as well as intermediate positions such as shown and illustrated in, for example, FIGS. 17, 26, and 27-29. The compression of the interbody spacer 10 varies the dimensions, shape, size, and volume of the hollow area 14, and facilitates insertion and initial placement of the interbody spacer 10 into the intervertebral space while in the compressed condition, and continued placement as the interbody spacer 10 is gradually expanded to the fully expanded position. When the interbody spacer is in the fully expanded position, the hollow area 14 is near a maximum possible size, maximizing the amount of material that may be deployed therein. Simultaneously, the rigid segments 12 are located within the intervertebral space so as to be adjoining areas of denser bone to minimize issues relating to subsidence and unwanted lordosis changes.

When the interbody spacer 10 is in the fully compressed position, certain of the rigid segments 12 are located on the ends of the interbody spacer 10. A distal segment 18 is that rigid segment 12 that will be first introduced into the patient, and will thus be most distal from the surgeon during the procedure. A proximal segment 20 is that rigid segment 12 that is most proximal the surgeon during the insertion procedure. All of the rigid segments 12 may include certain features to facilitate insertion and expansion of the interbody spacer 10, but in some embodiments, the distal segment 18 and the proximal segment 20 may include certain different features to facilitate insertion and expansion of the interbody spacer 10 than those features of other rigid segments 12. In other embodiments, rigid segments 12 immediately adjacent to the proximal segment 20 and/or the distal segment 20 may include certain different features to facilitate insertion and expansion of the interbody spacer 10 than those features of other rigid segments 12. Such features will be described in more detail with respect to certain of the Figures.

It may be noted from FIG. 16 that the hollow area 24 defined by the rigid segments 12 of the interbody spacer 10 is generally or roughly symmetrical in nature about a plane of symmetry 22. (In FIG. 16, the interbody spacer 10 is shown with the anterior portion of interbody spacer 10 up and the posterior portion of the interbody spacer 10 down.) This general symmetry of the expanded interbody spacer 10 minors the mediolateral symmetry of the vertebrae and intervertebral space. It may also be noted that the distal segment 18 and the proximal segment 20 are not located on the plane of symmetry 22 of the device, but are instead offset from the central plane of symmetry. This offset is to facilitate placement of the interbody spacer 10 using the selected surgical approach. The illustrated embodiment of FIG. 16, for example, might be suitable for use in the TLIF approach, whereby once the interbody spacer 10 is fully placed and expanded, the proximal segment 20 is correctly positioned posteriorly, off the center line of the spine, but on the center line of the access window created by the surgeon for the procedure.

The specific number, shape, and configuration of the various rigid segments 12 shown in FIG. 16 is intended to be illustrative only, and not limiting of the number, shape, and configuration of rigid segments 12 in interbody spacers 10 in accordance with embodiments of the invention. For example, the number, shape, and configuration of the various rigid segments 12 may be modified to accommodate for specific anatomical features of the patient, the desired site of fusion (e.g., L4-L5 as opposed to L5-S1), manufacturing preferences, and the desired surgical approach. For example, embodiments of the interbody spacer 10 may be used to reduce the invasiveness of surgery regardless of the surgical approach used, and the location of the distal segment 18 and the proximal segment 20 around the interbody spacer 10 may be varied to match the approach used so as to place the proximal segment 20 most proximal the surgeon on line with the chosen surgical approach. Even where the interbody spacer 10 is to be used for an identical approach, the number, shape, and configuration of the various rigid segments 12 may be varied, as may be noted by comparing them embodiment of FIG. 16 with the embodiment of FIG. 17, wherein the shape of the proximal segment 20 varies between embodiments.

The joints 16 may provide constrained motion between adjoining rigid segments 12. The joints 16 may be configured, for example, to allow only sufficient motion between adjoining rigid segments 12 to permit the adjoining rigid segments 12 to move the amount necessary for insertion and deployment of the interbody spacer 10. The joints 14 may be provided in certain embodiments by relatively flexible material extending between adjoining rigid segments 12, as illustrated in FIG. 17. In other embodiments, the joints 14 may be provided by a flexible member extending across multiple rigid segments 12, on which the rigid segments 12 are disposed (e.g. with the flexible member being disposed in a channel or other receiving element in the rigid segments 12.

Where the joints 14 are provided by relatively flexible material extending between adjoining rigid segments 12, the relatively flexible material may be provided in any of a variety of fashions. In one example, illustrated in FIG. 17, the relatively flexible material extending between adjoining rigid segments 12 is integrally formed with the adjoining rigid segments 12 as flexures 30. In some embodiments, the relatively flexible material (e.g., flexures 30) is integrally formed with the adjoining rigid segments 12 as part of the manufacturing process of the rigid segments 12, and may be made of a material similar to or identical to the material of the rigid segments 12. In such a case, the relative flexibility of the flexible region may be achieved by the dimensions provided to the material of the flexible region. For example, the flexible region can be made flexible by being made thin at the flexible region. In the embodiment illustrated in FIG. 17, the relatively flexible material forming the joints 16 includes two flexure divisions, or a split flexure 30 to modify its physical and performance characteristics, such as to increase flexibility without loss of strength, to reduce stress and force while increasing resistance to shear and tension, etc. In other embodiments, the relatively flexible material forming one or more of the joints 16 may include three or more flexure divisions.

Where the interbody spacer 10 is manufactured as a unitary device such as is illustrated in FIG. 17, the device may be manufactured using any suitable process. For example, the interbody spacer 10 may be manufactured by casting. As another alternative, the interbody spacer 10 may be manufactured using a 3D printing process. Any suitable current or future manufacturing process adapted to the material being used may be used to manufacture the interbody spacer 10 having integrally formed joints 16 and rigid segments 12. As a manufacturing option, the interbody spacer 10 may be manufactured in a partially deployed position (a position between the fully compressed position and the fully deployed position) to reduce stresses on the joints 16, and such a position is illustrated in FIG. 17.

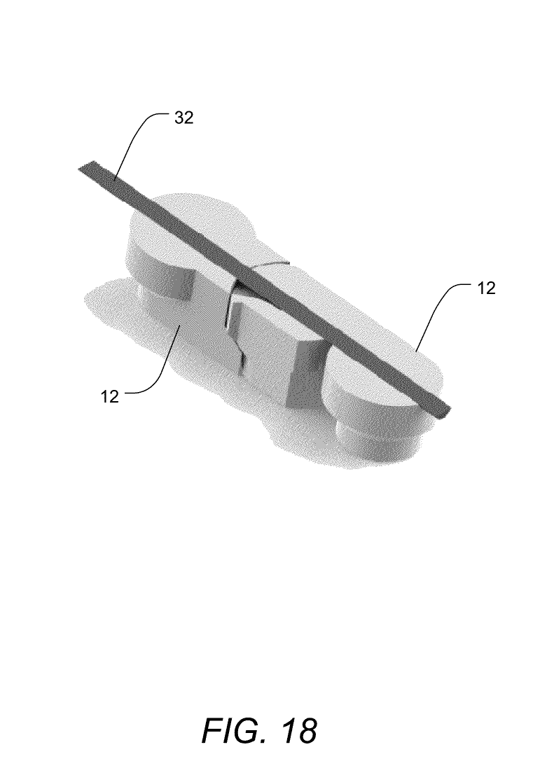

Alternatively, the flexible material or flexible region can be separately manufactured and can be attached to the rigid segments 12, using any suitable process or technique. In such a case, the material forming the relatively flexible region might differ from the material of the rigid segments 12. In some embodiments, as illustrated in FIG. 18, the relatively flexible region may be provided by a continuous flexible member 32 extending between multiple rigid segments 12. While FIG. 18 illustrates a single continuous flexible member 32 extending between adjoining rigid segments 12, more than one continuous flexible members 32 may be used (e.g., each rigid segment 12 may have two channels adapted to receive or receiving two continuous flexible members 32, or more channels adapted to receive or receiving more continuous flexible members 32). A nitinol wire is one example of a possible continuous flexible member 32.

One end of the continuous flexible member 32 is fixedly attached to one of the rigid segments 12 (such as to the distal segment 18 or the proximal segment 20), and the continuous flexible member 32 is passed through surface or internal channels on each adjoining rigid segment 12 until a desired arrangement has been made and the continuous flexible member can be attached at the end of the chain of rigid segments 12. Before or as such final attachment occurs, a proper amount of tension may be applied to the continuous flexible member so as to cause the interbody spacer 10 to have desired performance characteristics (e.g. a tendency to return to a desired native position, or a tendency to resist certain applications of forces in certain directions to a certain extent).

In ways such as this, an interbody spacer 10 might have a multisegmented region connected through the continuous flexible member 32, or the entire ring of the interbody spacer might be formed of one or more multisegmented regions connected through one or more continuous flexible members 32. In some embodiments, the interbody spacer 10 may include any combination of multisegmented regions with integrally formed flexible regions, multisegmented regions with separately formed and attached flexible regions, and multisegmented regions connected through a continuous flexible member 32. Depending on the manner in which the flexible regions are provided, they may be completely or partially internal to the interbody spacer 10, so as to reduce any risk of pinching or entrapping other objects between rigid segments 12.

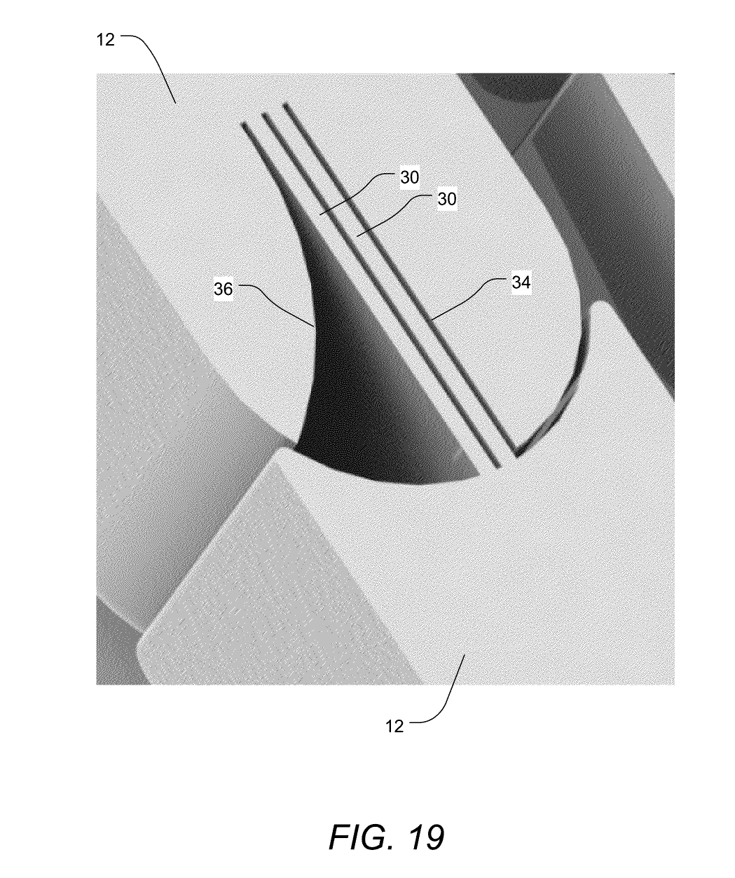

Regardless of the mechanism used to provide relatively flexible regions at the joints 16 between the rigid segments 12, the interbody spacer 10 may be manufactured with features to limit the range of motion of each joint 16 of the interbody spacer 10. For example, the range of motion may be limited to a range between approximately twenty and approximately one hundred twenty degrees. The range of motion may be limited using any satisfactory mechanism or method, but the range of motion is generally limited so as to prevent the interbody spacer 10 from unwantedly assuming configurations that will not be satisfactory either for insertion or for deployment of the interbody spacer 10. For example, FIG. 19 illustrates and enlarged view of one joint 16 of an interbody spacer 10 similar to that shown in FIG. 17. As may be seen, the adjoining rigid segment 12 includes a first flexure contact surface 34 and a second flexure contact surface 36.

The first flexure contact surface 34 and the second flexure contact surface 36 are adapted to engage the flexure 30 or flexures 30 (illustrated as a two-segment split flexure 30 in FIG. 19) so as to limit relative motion between the adjoining rigid segments 12. The first flexure contact surface 34 serves as a stop to prevent further clockwise rotation of the upper (as shown in FIG. 19) rigid segment 12 relative to the lower rigid segment 12. The second flexure contact surface 36 serves as a stop to prevent further counterclockwise rotation of the upper rigid segment 12 relative to the lower rigid segment 12. The respective contact surfaces may be straight or curved, as desired, to distribute stresses on the flexures 30 between rigid segments 12 along the whole flexures 30 as a point of maximal displacement is achieved.

While FIG. 19 illustrates principles applicable to reducing range of motion using an integral flexure 30, similar principles may be used where rigid segments 12 are disposed on one or more continuous flexible members 32. For example, a channel containing the continuous flexible member 32 may be broadened near the edge of some or all of the rigid segments, with each broadening being to an extent and with a curvature desired to permit a desired range of motion at the joint 16 at that edge.

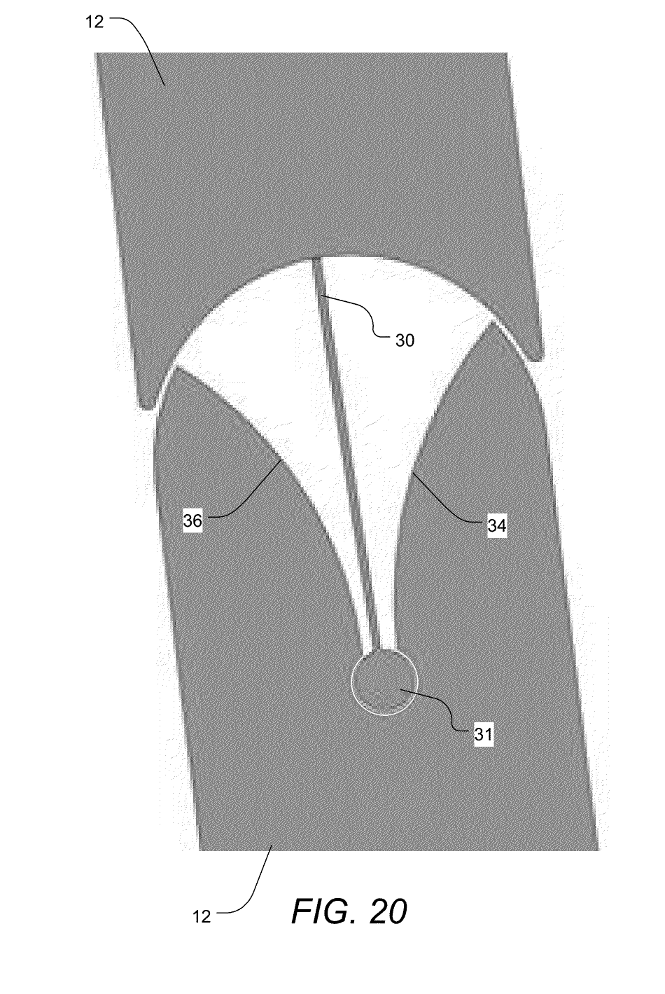

FIG. 20 also illustrates principles applicable to range of motion, and also illustrates an alternative manner of providing a flexible region at the joint 16 between adjacent rigid segments 12. In this embodiment, the flexible region at the joint 16 is still provided by a flexure 30, but the flexure 30 is only integrally formed with and/or fixedly attached to one of the two adjoining rigid segments 12. The other end of the flexure 30 includes or is attached to a pivot 31 that is disposed within a pivot receptacle of the other of the adjoining rigid segments 12. This arrangement of a pivot 31 and pivot receptacle allows the flexure 30 to more freely rotate around a portion of its range of motion, until the flexure 30 begins to impact the first flexure contact surface 34 or the second flexure contact surface 36. Such an arrangement may serve as another mechanism to reduce stress at the rigid-flexible interface. In another embodiment, both ends of the flexure 30 may be formed as pivots 31 inserted in pivot receptacles.

As another example of a mechanism to limit motion, motion between adjoining rigid segments 12 may be permitted until rigid portions of adjoining rigid segments 12 interact. Thus, instead motion being constrained by impact of a flexure 30 on a first flexure contact surface 34 or the second flexure contact surface 36, motion may be constrained by a contact surface of one rigid segment 12 impacting a contact surface of an adjacent rigid segment 12.

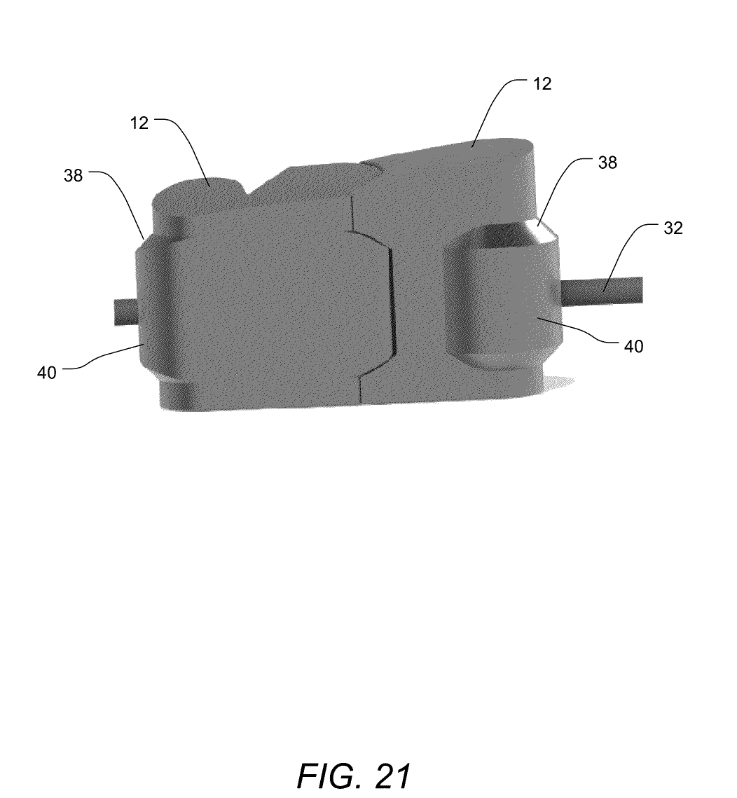

One or more of the rigid segments 12 may be manufactured with one or more contacting surfaces adapted to contact adjoining rigid segments 12 while reducing forces experienced by the flexible segments, regions, or members extending between the adjoining rigid segments 12. For example, as illustrated in FIG. 21, the rigid segments may be manufactured with one or more conical surfaces 38 and/or cylindrical surfaces 40 adapted to reduce the compressive, torsional, and shear forces experienced by the continuous flexible member 32. Taking such forces into account reduces the chances that the continuous flexible member 32 will fail during insertion and deployment of the interbody spacer 10.

Regardless of the specific method of manufacture of the interbody spacer 10 or the specific components of the interbody spacer 10 (such as the components providing the flexible regions), one purpose of the interbody spacer 10 is to provide an implant that can assume a narrow compressed aspect, while reliably being deployed to a fully deployed state. For example the interbody spacer 10 may achieve a lateral compression to have an insertion width that is only approximately 20% to 30%, e.g., 25%, of the deployed width of the interbody spacer 10. This greatly reduced insertion width allows the interbody spacer 10 to be used as a large implant while being inserted in a small surgical window, reducing the invasiveness of surgery while increasing the effectiveness of the device.

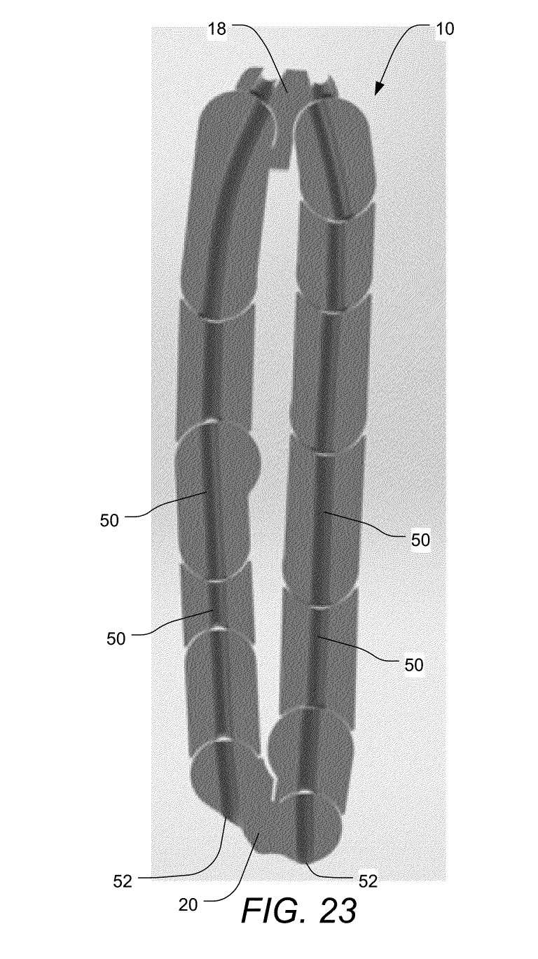

The interbody spacer 10 and the rigid segments 12 include features to assist the surgeon in achieving and maintaining the narrow insertion aspect as shown in FIG. 22 and in reliably deploying the interbody spacer 10 to the fully deployed aspect illustrated in FIG. 16. First, many or all of the rigid segments 12 may include one or more insertion channels 50 as shown in FIG. 23. The insertion channels 50 of the rigid segments 12 align when the interbody spacer 10 is compressed into its narrow insertion aspect, as illustrated in FIG. 23. In this aspect, the insertion channels 50 terminate at insertion openings 52 on the proximal segment 20 (as illustrated in FIG. 23) or on the rigid segments 12 immediately adjacent the proximal segment 20 (as illustrated in FIG. 17).

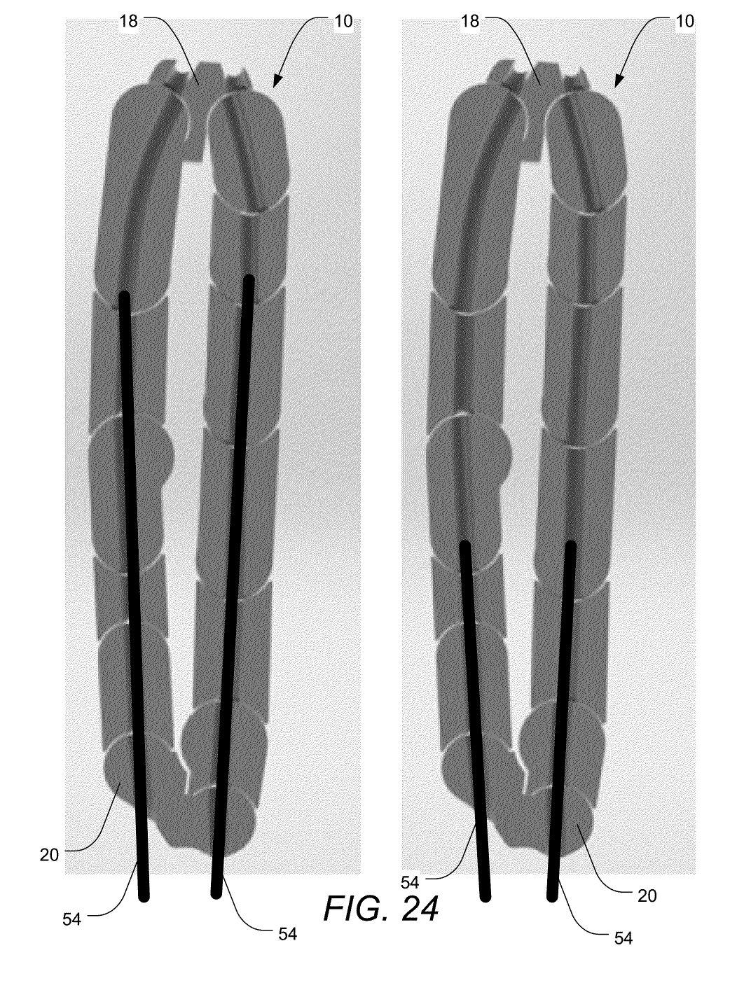

When the interbody spacer 10 is compressed into its narrow insertion aspect so that the insertion channels align, one or more insertion rods 54 may be inserted into the insertion openings 52 and into the insertion channels, thereby locking rotation at each joint 16 through which the insertion rods 54 pass, as illustrated in FIG. 24. The interbody spacer 10 may be delivered to the surgeon in its narrow insertion aspect with the insertion rods 54 already fully inserted, or it may be delivered in a partially or fully relaxed aspect and the surgeon then compresses the interbody spacer 10 and inserts the insertion rods 54. During the insertion procedure, the surgeon selectively withdraws the insertion rods 54 a certain amount at times to free certain joints 16 to rotate as limited by any rotation-limiting features to permit partial deployment of the interbody spacer 10, additional insertion of the interbody spacer 10, and eventually full deployment of the interbody spacer 10, as will be described.

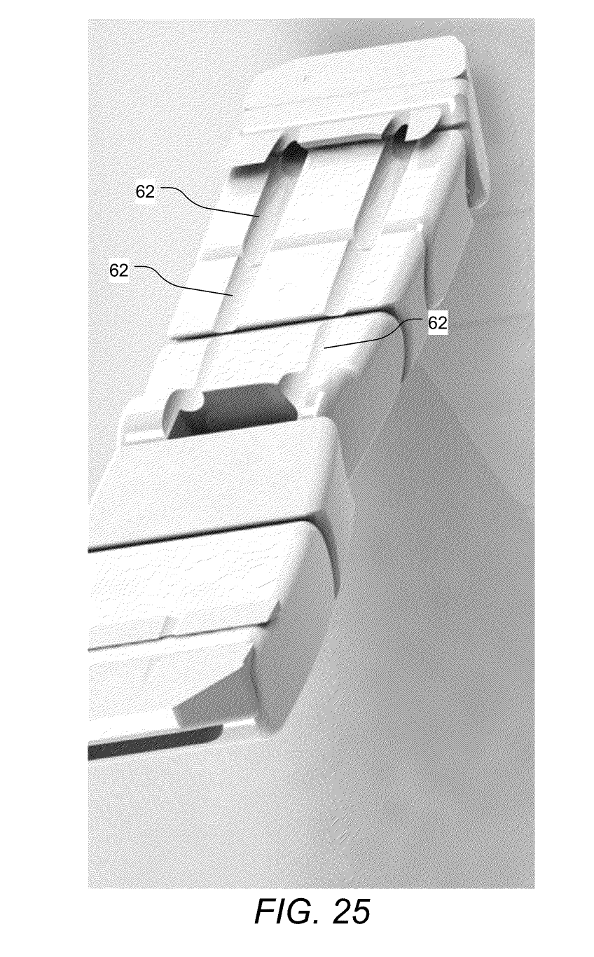

While the interbody spacer 10 may partially or even fully deploy based on natural tensions imparted by the flexible regions between rigid segments 12, embodiments of the interbody spacer 10 include features to allow a surgeon to impart deploying forces to the interbody spacer. Specifically, the distal segment 18 may be provided with a pull rod attachment point 60 as shown in adapted to receive a pull rod, and the proximal segment 20 may be provided with a pull rod channel, as shown in FIG. 17. As necessary, one or more pull rod channels 62 may be formed on inwardly facing surfaces of other rigid segments 12 to permit the pull rod to be attached to the pull rod attachment point 60 while the interbody spacer 10 is in the narrow insertion aspect, as illustrated in FIG. 25. While the description herein refers to a single pull rod, embodiments of the invention may utilize more than one pull rod, pull rod attachment point 60, and/or pull rod channel 62, as is illustrated in FIGS. 25, 28, and 29. The pull rod may be attached to the pull rod attachment point 60 using any acceptable method of attachment, such as by permanent attachment, by threaded (screw-type) attachment, or the like.

The interbody spacer 10 is used in a normal interbody spinal fusion procedure as follows.

The surgeon surgically accesses the site and prepares the intervertebral space as normal, removing the damaged intervertebral disc to an extent to allow for placement of the fully deployed interbody spacer 10. Then, the surgeon introduces the interbody spacer 10 at the surgical site, with the interbody spacer 10 in its narrow insertion aspect (e.g., FIG. 22), with the insertion rods 54 fully inserted into the insertion channels 50 (e.g. left view of FIG. 24) and with the pull rod attached to the pull rod attachment point 60 and threaded through the pull rod channel 62.

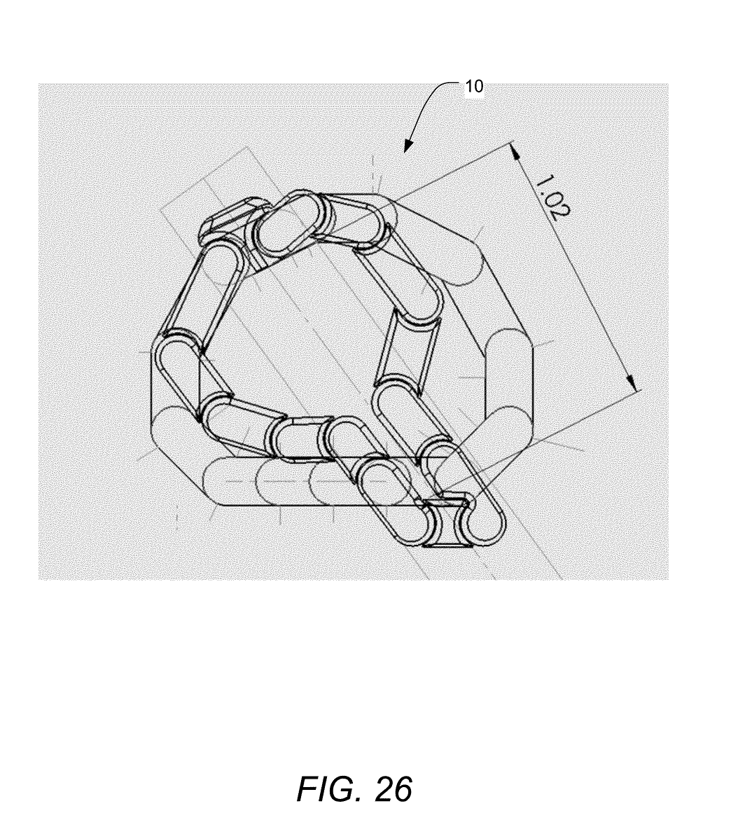

The interbody spacer 10 is inserted, distal segment 18 first, through the surgical access window and into the intervertebral space until the distal segment 18 nears or reaches the most distal cleared portion of the intervertebral space. At this point, the insertion rod 54 or insertion rods 54 are partially withdrawn (e.g. right view of FIG. 24), whereupon the distal portion of the interbody spacer 10 begins to deploy, either under natural forces (applied by the flexures 30, continuous flexible member 32, or other structure providing the flexible regions between rigid segments 12) or alternatively or additionally by way of the surgeon pulling on the pull rod. The result is partial deployment of the distal portion of the interbody spacer, as is illustrated in FIG. 26, which shows an overlay of a partially deployed interbody spacer 10 over a fully deployed interbody spacer 10.

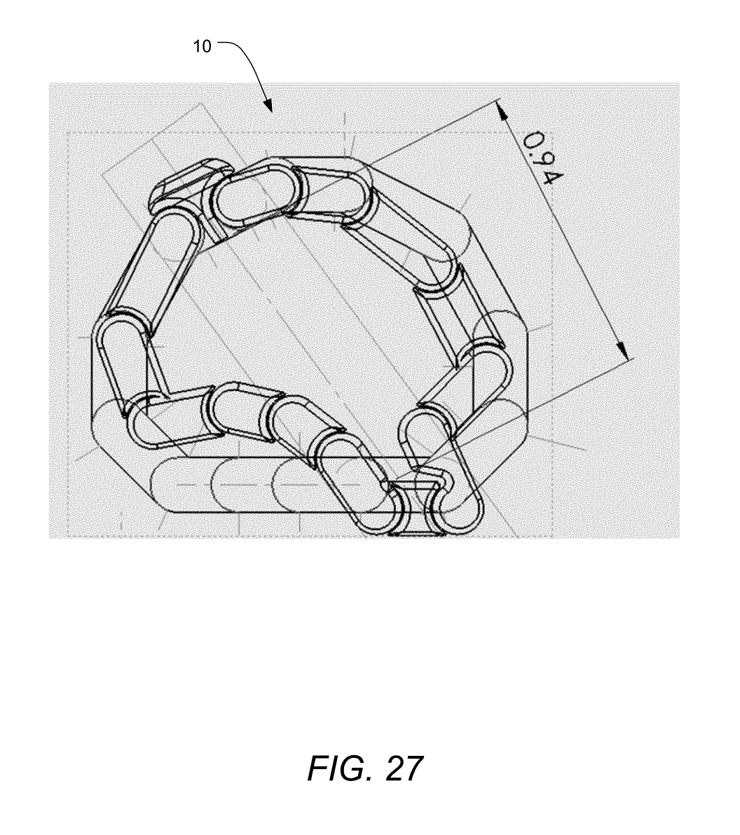

During or after partial deployment, the surgeon more fully inserts the interbody spacer 10 into the intervertebral space, as more room has been made by the partial retraction of the distal segment 18. The surgeon then repeats or continues the process of withdrawing the insertion rod 54 or insertion rods 54 and pulling on the pull rod to further deploy the interbody spacer, as illustrated in FIG. 27. Eventually, the surgeon fully removes the insertion rod 54 or insertion rods 54, and fully deploys the interbody spacer 10 fully within the intervertebral space by pulling on the pull rod. The surgeon may adjust placement and deployment of the interbody spacer 10 as necessary through the surgical access window (or windows) and then detaches the pull rod or cuts off the pull rod at the proximal surface of the interbody spacer 10.

The hollow area 14 of the interbody spacer 10 can then be filled with any desirable material, as discussed above, and the surgeon may also surround all or a portion of any accessible portions of the interbody spacer 10 with such materials, and then the surgery may be completed as with conventional spinal fusion surgeries, including the placement of posterior supports (pedicle screws, bars, and/or plates) as necessary.

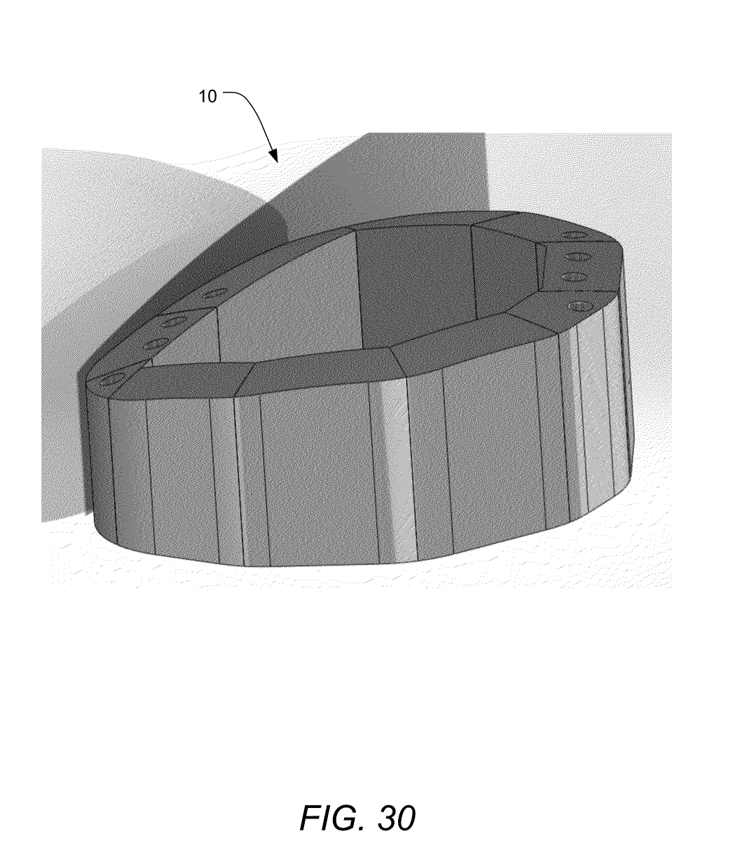

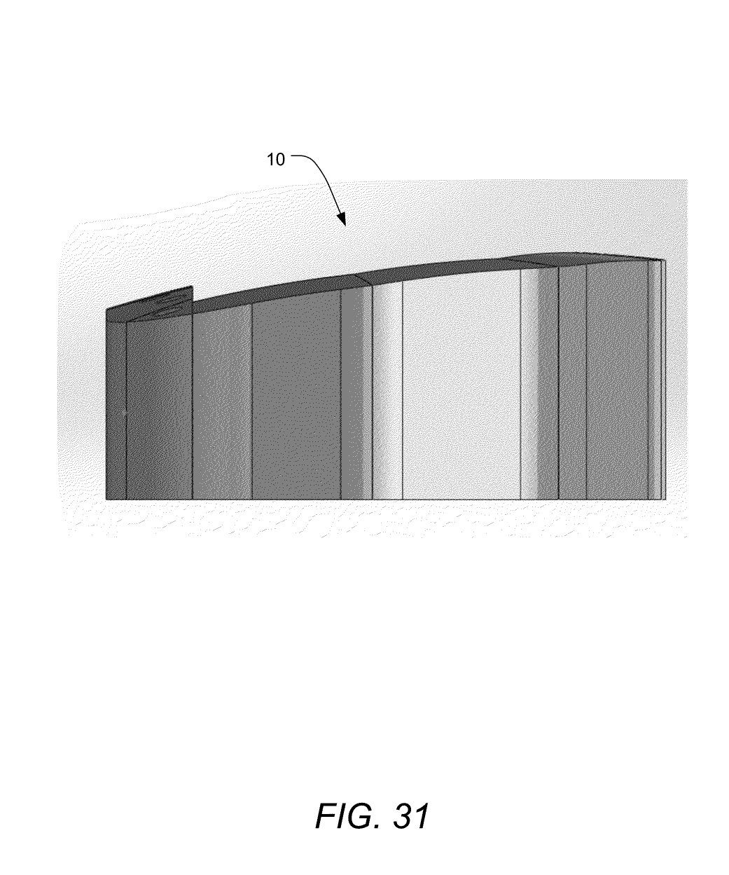

FIGS. 28 and 29 show partially disassembled views of embodiments of the interbody spacer 10 to illustrate functioning of the insertion channels 50, insertion openings 52, and insertion rods 54, as well as functioning of the pull rod attachment points 60 and pull rod channels 62. FIGS. 30 and 31 show perspective and side views of an embodiment of a deployed interbody spacer 10. These Figures illustrate that the interbody spacer 10 may have a non-constant height so as to match the desired intervertebral volume for proper lordosis. While not illustrated in the Figures, one or more of the superior or inferior surfaces of one or more rigid segments 12 of the interbody spacer 10 may include one or more protrusions or other features to minimize unwanted motion of the interbody spacer 10 once placed in the intervertebral space, although the large size of the interbody spacer 10 will generally minimize such movement in any event.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. An expandable interbody spacer for use in spinal fusion procedures, comprising:
a plurality of rigid segments connected by flexible connections to form a ring encompassing and defining a hollow central area of variable dimensions.

2. The expandable interbody spacer as recited in claim 1, wherein the flexible connections between the plurality of rigid segments comprise flexible regions formed between the rigid segments.

3. The expandable interbody spacer as recited in claim 2, wherein the flexible regions formed between the rigid segments are integrally formed with the rigid segments.

4. The expandable interbody spacer as recited in claim 2, wherein one or more of the flexible regions formed between the rigid segments comprise a plurality of flexure divisions extending between adjacent rigid segments.

5. The expandable interbody spacer as recited in claim 2, wherein one or more of the flexible regions formed between the rigid segments comprise a flexure extending between adjacent rigid segments.

6. The expandable interbody spacer as recited in claim 5, wherein the flexure comprises a pivot disposed in a pivot receptacle of one of the rigid segments.

7. The expandable interbody spacer as recited in claim 5, wherein a range of motion between adjoining rigid segments is constrained by rigid surfaces of each of the adjoining rigid segments.

8. The expandable interbody spacer as recited in claim 5, wherein a rigid segment adjacent the flexure comprises one or more surfaces adapted to define a maximum range of motion between the adjacent rigid segments.

9. The expandable interbody spacer as recited in claim 8, wherein the maximum range of motion between the adjacent rigid segments comprises between approximately twenty and approximately one hundred and twenty degrees.

10. The expandable interbody spacer as recited in claim 1, wherein the flexible connections between the plurality of rigid segments comprise a continuous flexible member extending between and comprising multiple flexible connections of the expandable interbody spacer.

11. The expandable interbody spacer as recited in claim 10, wherein the continuous flexible member passes through one or more channels formed in one or more of the rigid segments.

12. The expandable interbody spacer as recited in claim 10, wherein the continuous flexible member extends around the entire interbody spacer.

13. The expandable interbody spacer as recited in claim 1, wherein the rigid segments comprise stability channels adapted to receive a stability rod therein, whereby insertion of the stability rod therein serves to limit motion between adjacent rigid segments.

14. The expandable interbody spacer as recited in claim 1, wherein one or more of the rigid segments comprises a conical contact surface adapted to contact an adjacent rigid segment.

15. The expandable interbody spacer as recited in claim 1, wherein one or more of the rigid segments comprises a cylindrical surface adapted to contact an adjacent rigid segment.

16. The expandable interbody spacer as recited in claim 1, wherein the ring is adapted such that the hollow central area can be made narrow during initial insertion of the interbody spacer and then expanded horizontally as the interbody spacer is fully inserted and placed in an intervertebral space between adjoining vertebrae.

17. The expandable interbody spacer as recited in claim 16, wherein one or more of the rigid segments comprises a pull channel adapted to permit application of a pulling force to the interbody spacer to expand the interbody spacer.

18. The expandable interbody spacer as recited in claim 1, wherein the hollow central area is adapted to receive a material selected from the group consisting of:
a bone graft material;
an osteoinductive material; and
an osteoconductive material.

19. An expandable interbody spacer for use in spinal fusion procedures, comprising:
a plurality of rigid segments arranged to form a ring defining a hollow central area;
a plurality of flexible connections formed by flexible members extending between adjacent rigid segments such that the ring can be deformed to modify dimensions of the hollow central area.

20. The expandable interbody spacer as recited in claim 19, wherein the flexible members formed between the rigid segments are integrally formed with the rigid segments.

21. The expandable interbody spacer as recited in claim 19, wherein one or more of the flexible regions formed between the rigid segments comprise a plurality of flexure divisions extending between adjacent rigid segments.

22. An expandable interbody spacer for use in spinal fusion procedures, comprising:
a plurality of rigid segments arranged to form a ring defining a hollow central area;
a flexible member extending around the ring and between adjacent rigid segments such that the ring can be deformed to modify dimensions of the hollow central area.
